(19) United States
(12) Patent Application Publication
(10) Pub. No.: US 2025/0370094 A1
(43) Pub. Date: Dec. 4, 2025

(54) RADAR SYSTEM AND SIGNAL PROCESSING METHOD FOR THE SAME

(71) Applicant: Murata Manufacturing Co., Ltd., Nagaokakyo-shi (JP)

(72) Inventor: Daichi UEKI, Nagaokakyo-shi (JP)

(73) Assignee: Murata Manufacturing Co., Ltd., Nagaokakyo-shi (JP)

(21) Appl. No.: 19/302,158

(22) Filed: Aug. 18, 2025

Related U.S. Application Data
(63) Continuation of application No. PCT/JP2024/001026, filed on Jan. 16, 2024.

(30) Foreign Application Priority Data
Apr. 5, 2023 (JP) 2023-061236

(51) Int. Cl. G01S 7/35; G01S 13/32; G01S 13/82; G01S 13/88

(52) U.S. Cl. CPC G01S 7/358; G01S 13/32; G01S 13/82; G01S 13/88

(57) ABSTRACT

A radar system includes a first device that acquires a displacement of the subject of displacement acquisition by transmitting and receiving a radio wave, and a second device installed at the subject capable of transmitting to and receiving from the first device. The second device has a first mode in which it reradiates a received radio wave and a second mode in which it delays a phase of a received radio wave before reradiating. The first device acquires first IQ information while the second device is operating in the first mode and second IQ information while the second device is operating in the second mode. The first device then calculates a direct-current component of the first IQ information and generates third IQ information by removing the direct-current component from the first IQ information.

### CROSS-REFERENCE TO RELATED APPLICATIONS

The present application is a continuation of International Application No. PCT/JP2024/001026, filed Jan. 16, 2024, which claims priority to Japanese patent application JP 2023-061236, filed Apr. 5, 2023, the entire contents of each of which being incorporated herein by reference.

### TECHNICAL FIELD

The present disclosure relates to a radar system and a signal processing method for the same.

### BACKGROUND ART

In the related art, a configuration has been disclosed in which biological information on a human body is acquired by using a radio wave sensor, such as a RAdio Detection And Ranging (RADAR) (for example, Patent Documents 1 and 2). Patent document 2 discloses a configuration in which information on a distance to a body surface of a living body obtained by processing an I signal obtained by multiplying a signal of an electromagnetic wave and a signal of a reflected wave and a Q signal obtained by delaying the I signal by a predetermined phase is output, a reception intensity of the reflected wave based on a diameter of a circle drawn by a signal point obtained by developing the I signal and the Q signal in a complex plane is output, and an phase change amount of the reflected wave based on a displacement angle of a range where the signal point is displaced on the circle with respect to a center of the circle is output.

### CITATION LIST

### Patent Document

Patent Document 1: Japanese Unexamined Patent Application Publication No. 2018-202921

Patent Document 2: Japanese Unexamined Patent Application Publication No. 2020-146235

### SUMMARY

### Technical Problems

In a reception signal, a direct-current component other than a signal corresponding to biological information is included. This direct-current component includes, for example, a reflected component from a stationary object or the like other than a human body. If this direct-current component is large, there is a possibility of being unable to acquire a minute displacement component (biological information and so forth), such as a body surface displacement of a relatively small human body, relative to the direct-current component with high accuracy.

The present disclosure has been made in view of the above and aims to provide a radar system that can acquire a minute displacement component of a subject of displacement acquisition with high accuracy, and a signal processing method for the same.

### Solutions to Problems

A radar system according to an aspect of the present disclosure includes a first device that acquires a displacement of a subject of displacement acquisition by using a reception wave, and a second device that is installed at the subject of displacement acquisition and is disposed within a range where the second device can transmit and receive a radio wave to and from the first device. The second device has a first mode in which the second device reradiates a received radio wave, and a second mode in which the second device delays a phase of a received radio wave and reradiates the received radio wave. The first device calculates, by using first information acquired in a first period during which the second device is operating in the first mode and second information acquired in a second period during which the second device is operating in the second mode, a direct-current component of the first information, and generates third information obtained by removing the direct-current component from the first information.

In this configuration, third information obtained by removing a direct-current component included in a reflected-wave component from a stationary object other than the subject of displacement acquisition can be obtained. This enables a minute displacement component of the subject of displacement acquisition to be acquired with high accuracy.

A signal processing method for a radar system according to an aspect of the present disclosure is a signal processing method for a radar system including a first device that acquires a displacement of a subject of displacement acquisition, and a second device that is installed at the subject of displacement acquisition and is disposed within a range where the second device can transmit and receive a radio wave to and from the first device. The second device has a first mode in which the second device reradiates a received radio wave, and a second mode in which the second device delays a phase of a received radio wave and reradiates the received radio wave. The signal processing method includes, with the first device, a first step of generating first information by using a radio wave received in a first period during which the second device is operating in the first mode, a second step of generating second information by using a radio wave received in a second period during which the second device is operating in the second mode, a third step of calculating a direct-current component of the first information by using the first information and the second information, and a fourth step of generating third information obtained by removing the direct-current component from the first information.

In this configuration, third information obtained by removing a direct-current component included in a reflected-wave component from a stationary object other than the subject of displacement acquisition can be obtained. This enables a minute displacement component of the subject of displacement acquisition to be acquired with high accuracy.

### Advantageous Effects

The present disclosure can provide the radar system that can acquire a minute displacement component of the subject of displacement acquisition with high accuracy, and the signal processing method for the same.

### BRIEF DESCRIPTION OF DRAWINGS

FIG. 1 is a block diagram illustrating a schematic configuration of a radar system according to an embodiment.

FIG. 2A is a block diagram illustrating a first specific example of a phase shifter of a second device.

FIG. 2B is a block diagram illustrating a second specific example of the phase shifter of the second device.

FIG. 3A is a first diagram illustrating a relationship between a transmission wave of a first device and a switching timing in the phase shifter of the first specific example.

FIG. 3B is a second diagram illustrating a relationship between a transmission wave of the first device and a switching timing in the phase shifter of the first specific example.

FIG. 4A is a first diagram illustrating a relationship between a transmission wave of the first device and a switching timing in the phase shifter of the second specific example.

FIG. 4B is a second diagram illustrating a relationship between a transmission wave of the first device and a switching timing in the phase shifter of the second specific example.

FIG. 5 is a complex plane diagram illustrating an example of a relationship between first IQ information and second IQ information.

FIG. 6 is a flowchart illustrating a specific example of a process in the radar system according to the embodiment.

FIG. 7 is a sub-flowchart illustrating an example of an IQ information generation process.

FIG. 8 is a sub-flowchart illustrating an example of a direct-current component removal process.

FIG. 9 is a sub-flowchart illustrating an example of a displacement calculation process.

FIG. 10A is a first conceptual diagram illustrating a specific example of a direct-current component calculation process in a complex plane.

FIG. 10B is a first conceptual diagram illustrating coordinates of post-direct-current component removal first IQ information and second IQ information in a complex plane.

FIG. 11A is a second conceptual diagram illustrating a specific example of the direct-current component calculation process in a complex plane.

FIG. 11B is a second conceptual diagram illustrating coordinates of post-direct-current component removal first IQ information and second IQ information in a complex plane.

FIG. 12 is a flowchart illustrating a modification of the process in the radar system according to the embodiment.

FIG. 13 is a sub-flowchart illustrating an example of an IQ information generation process according to the modification.

### DESCRIPTION OF EMBODIMENTS

A radar system according to an embodiment and a signal processing method for the same will be described in detail below with reference to the drawings. Note that the embodiment is not intended to limit the present disclosure.

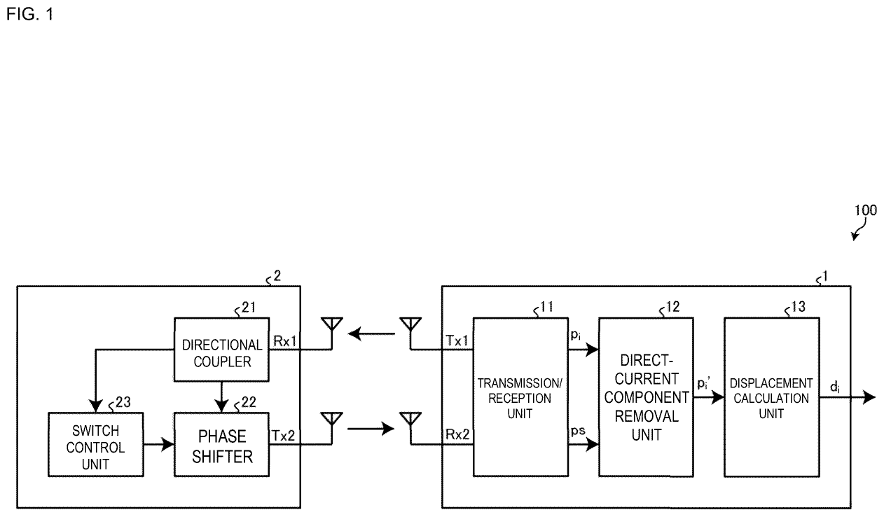

FIG. 1 is a block diagram illustrating a schematic configuration of a radar system according to the embodiment. A radar system 100 according to the Embodiment includes a first device 1 and a second device 2.

The first device 1 is a so-called RAdio Detection And Ranging (RADAR) device. Examples of a radar device include a Frequency Modulated Continuous Wave (FMCW) radar, a Doppler radar, and a pulse radar. In the present disclosure, the first device 1 includes a transmission/reception unit 11, a direct-current component removal unit 12, and a displacement calculation unit 13. As used herein, “unit” refers to circuitry that may be configured via the execution of computer readable instructions, and the circuitry may include one or more local processors (e.g., CPU's), and/or one or more remote processors, such as a cloud computing resource, or any combination thereof.

The second device 2 is installed at a target regarded as a subject of displacement acquisition in the radar system 100 (specifically, a human body, or a target to which a body surface displacement of a human body propagate, such as a seat in a vehicle or a bed). In the present disclosure, the second device 2 includes a directional coupler 21, a phase shifter 22, and a switch control unit 23.

The second device 2 is disposed within a range where the second device 2 can transmit and receive a radio wave to and from the first device 1 and has a first mode in which the second device 2 reradiates a received radio wave, and a second mode in which the second device 2 delays the phase of a received radio wave and reradiates the received radio wave.

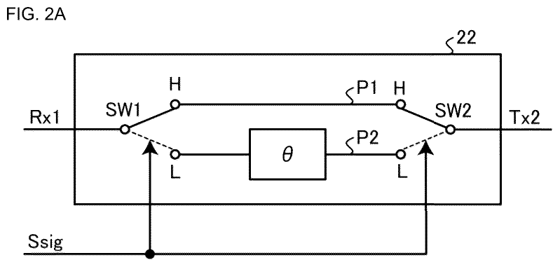

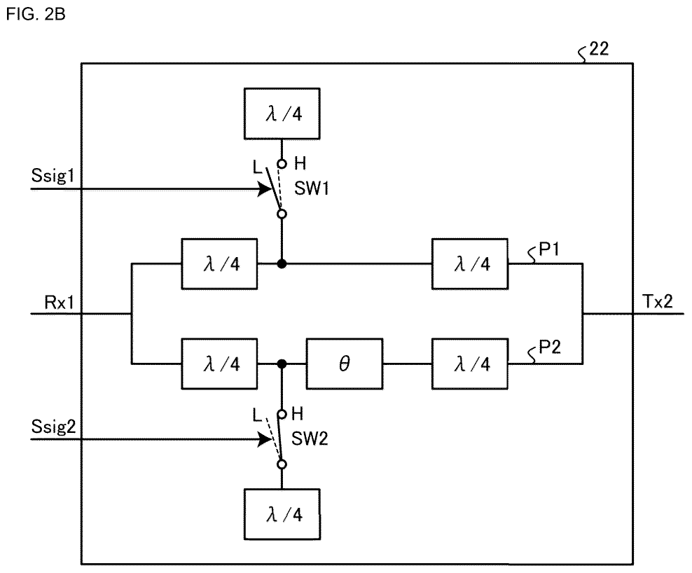

A radio wave received by the second device 2 is input to the directional coupler 21 as a reception wave Rx1. The directional coupler 21 outputs the reception wave Rx1 to the phase shifter 22. FIG. 2A is a block diagram illustrating a first specific example of the phase shifter of the second device 2. FIG. 2B is a block diagram illustrating a second specific example of the phase shifter of the second device 2.

The phase shifter 22 includes switch circuits SW1 and SW2 that switch between a path P1 that reradiates the reception wave Rx1 as a radiation wave Tx2 and a path P2 that reradiates a radiation wave Tx2 obtained by delaying the phase of the reception wave Rx1. In a configuration illustrated in FIG. 2B, in a path to which an open stub with a λ/4-length is connected, impedance at a connection point of the open stab for a signal with a wavelength λ is infinite. Assuming that a wavelength λ of a transmission frequency of the first device 1 (the frequency of a radiation wave Tx1) is one period (2π), a phase shift amount (amount of phase delay) θ in the phase shifter 22 is given in a range of 0<θ<2π. Within this range, the phase shift amount (amount of phase delay) θ is predetermined by the particular phase shifter.

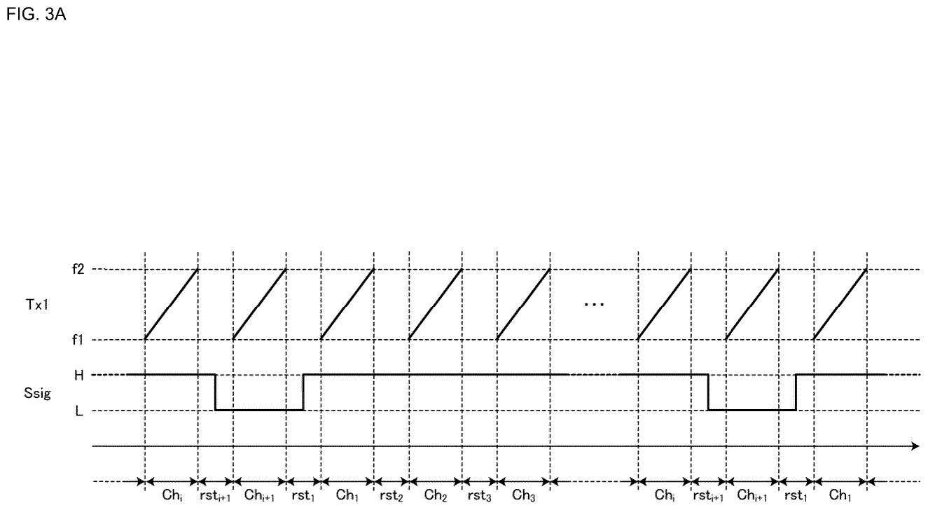

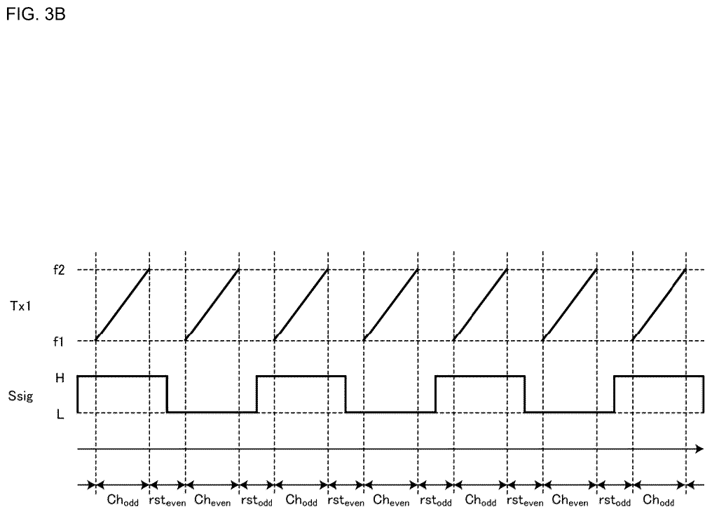

The switch circuits SW1 and SW2 are controlled by the switch control unit 23. FIG. 3A is a first diagram illustrating a relationship between a transmission wave of the first device and a switching timing in the phase shifter of the first specific example.

FIG. 3A illustrates an example of a radiation wave Tx1 in a case where the first device 1 is an FMCW radar. In this example, the first device 1 transmits a chirp signal Ch that modulates linearly in frequency from a frequency f1 to a frequency f2 on a predetermined cycle with a reset period rst being provided. When a transmission cycle of the chirp signal Ch is, for example, 1 ms, the chirp signal Ch modulates linearly in frequency from the frequency f1 to the frequency f2, for example, in 10 μs to 50 μs.

In a configuration of the first specific example illustrated in FIG. 2A, the switch control unit 23 controls a switch control signal Ssig from “L” to “H” in a reset period rst₁ after receiving a chirp signal Chᵢ₊₁ and controls the switch control signal Ssig from “H” to “L” in a reset period rstᵢ₊₁ after receiving a chirp signal Chᵢ. Thus, chirp signals Ch₁, Ch₂, Ch₃, . . . , and Chᵢ are reradiated as a radiation wave Tx2 (first mode), and the chirp signal Chᵢ₊₁ is reradiated as a radiation wave Tx2 with the phase delayed by the phase shift amount θ (second mode). For example, when a cycle on which “L” of the switch control signal Ssig in the switch control unit 23 is selected is ten times the transmission cycle of the chirp signal Ch (a switch control frequency is 100 Hz), i=9 holds.

FIG. 3B is a second diagram illustrating a relationship between a transmission wave of the first device and a switching timing in the phase shifter of the first specific example. In FIG. 3B, a switching cycle of the switch control signal in the switch control unit 23 is twice the transmission cycle of the chirp signal Ch (the switch control frequency is 500 Hz). In this case, for example, a chirp signal Ch_{odd} is reradiated as a radiation wave Tx2 in an odd-numbered cycle of the chirp signal Ch (first mode), and a chirp signal Chₑᵥₑₙ is reradiated as a radiation wave Tx2 with the phase delayed by the phase shift amount θ in an even-numbered cycle of the chirp signal Ch (second mode).

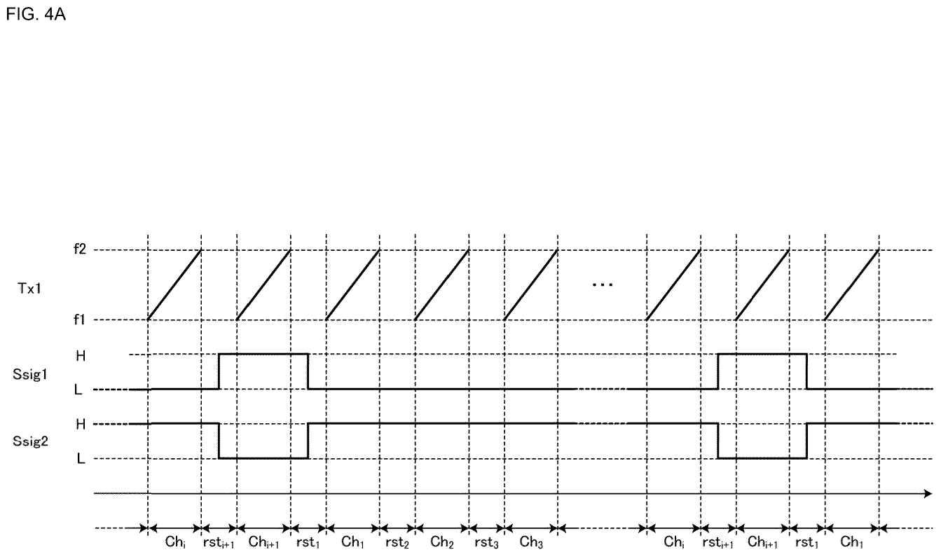

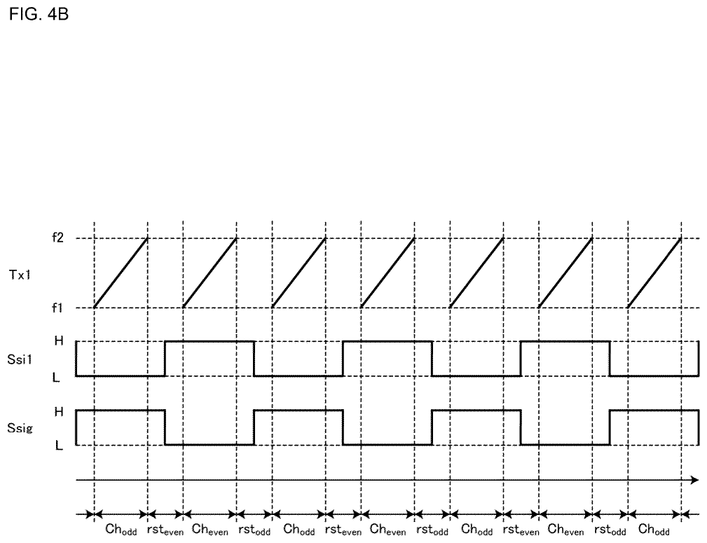

FIG. 4A is a first diagram illustrating a relationship between a transmission wave of the first device and a switching timing in the phase shifter of the second specific example.

In a configuration of the second specific example illustrated in FIG. 2B, the switch control unit 23 controls a switch control signal Ssig1 from “H” to “L” and controls a switch control signal Ssig2 from “L” to “H” in a reset period rst₁ after receiving a chirp signal Chᵢ₊₁. Furthermore, the switch control unit 23 controls the switch control signal Ssig1 from “L” to “H” and controls the switch control signal Ssig2 from “H” to “L” in a reset period rstᵢ₊₁ after receiving a chirp signal Chᵢ. Thus, chirp signals Ch₁, Ch₂, Ch₃, . . . , and Chᵢ are reradiated as a radiation wave Tx2 (first mode), and the chirp signal Chᵢ₊₁ is reradiated as a radiation wave Tx2 with the phase delayed by the phase shift amount θ (second mode). For example, in the switch control unit 23, when a cycle on which “H” of the switch control signal Ssig1 is selected and the cycle on which “L” of the switch control signal Ssig2 are ten times the transmission cycle of the chirp signal Ch (the switch control frequency is 100 Hz), i=9 holds.

FIG. 4B is a second diagram illustrating a relationship between a transmission wave of the first device and a switching timing in the phase shifter of the second specific example. In FIG. 4B, a switching cycle of the switch control signals Ssig1 and Ssig2 in the switch control unit 23 is twice the transmission cycle of the chirp signal Ch (the switch control frequency is 500 Hz). In this case, for example, a chirp signal Ch_{odd} is reradiated as a radiation wave Tx2 in an odd-numbered cycle of the chirp signal Ch (first mode), and a chirp signal Chₑᵥₑₙ is reradiated as a radiation wave Tx2 with the phase delayed by the phase shift amount θ in an even-numbered cycle of the chirp signal Ch (second mode).

Although FIG. 2B illustrates the configuration in which the switch control unit 23 outputs the switch control signals Ssig1 and Ssig2 to control the respective switch circuits SW1 and SW2, the switch control unit 23 may output a switch control signal Ssig to control the switch circuit SW2 and may control the switch circuit SW1 by using a signal obtained by logically inverting the switch control signal Ssig. In this way, control logic of the switch control signal Ssig in the switch control unit 23 is the same as in FIG. 3A or 3B.

The first device 1 receives a radiation wave Tx2 from the second device 2. Here, a reception wave Rx2 received by the first device 1 includes, in addition to the radiation wave Tx2, a reflected-wave component from a stationary object, e.g., furniture, walls, or other static items, within the radar's field of view, or the like other than a target regarded as a subject of displacement acquisition in the radar system 100. A reflected wave from such a stationary object includes a direct-current component unrelated to a minute displacement component (alternating-current component) in the subject of displacement acquisition.

In the present disclosure, the direct-current component removal unit 12 generates, by using first IQ information (first information) acquired in a first period during which the second device 2 is operating in the first mode and second IQ information (second information) acquired in a second period during which the second device 2 is operating in the second mode, third IQ information (third information) obtained by removing a direct-current component of the first IQ information. Thus, third IQ information obtained by removing a direct-current component included in a reflected-wave component from a stationary object or the like other than the target regarded as the subject of displacement acquisition in the radar system 100 can be obtained.

Specifically, the transmission/reception unit 11 generates an I signal in phase with the radiation wave Tx1 and a Q signal in quadrature with the radiation wave Tx1 by using the reception wave Rx2. The I signal and the Q signal can be defined by coordinates in a complex plane defined by a real axis (Re axis) and an imaginary axis (Im axis).

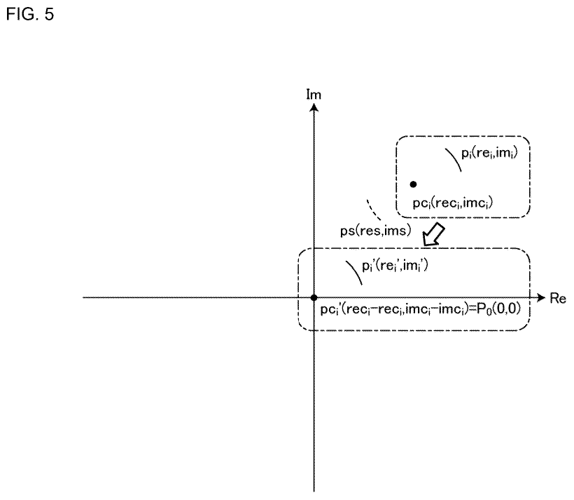

FIG. 5 is a complex plane diagram illustrating an example of a relationship between first IQ information and second IQ information. The transmission/reception unit 11 takes an I signal reᵢ and a Q signal imᵢ generated in the first mode as first IQ information in the complex plane and takes an I signal res and a Q signal ims acquired in the second mode as second IQ information in the complex plane. In FIG. 5, in the complex plane defined by the real axis (Re axis) and the imaginary axis (Im axis), coordinates of the first IQ information are pᵢ(reᵢ, imᵢ), and coordinates of the second IQ information are ps(res, ims). Furthermore, coordinates of a direct-current component of the first IQ information pᵢ are pcᵢ(recᵢ, imcᵢ), and coordinates of the third IQ information are pᵢ′(reᵢ′, imᵢ′).

In the following description, the first IQ information defined by the coordinates pᵢ(reᵢ, imᵢ) in the complex plane is also referred to as “first IQ information pᵢ”, and the second IQ information defined by the coordinates ps(res, ims) in the complex plane is also referred to as “second IQ information ps”. Furthermore, the direct-current component of the first IQ information pᵢ defined by the coordinates pcᵢ(recᵢ, imcᵢ) in the complex plane is also simply referred to as “direct-current component pcᵢ”, and the third IQ information defined by the coordinates pᵢ′(reᵢ′, imᵢ′) in the complex plane is also referred to as “third IQ information pᵢ′”.

FIG. 5 illustrates an example in which, when the wavelength of the transmission frequency of the first device 1 (the frequency of the radiation wave Tx1) is λ, the phase shift amount (amount of phase delay) θ in the second mode is λ/2. In this example, the direct-current component pcᵢ included in the first IQ information pᵢ can be calculated as a mean value of the first IQ information pᵢ and the second IQ information ps. At this time, the coordinates pcᵢ(recᵢ, imcᵢ) of the direct-current component pcᵢ in the complex plane are expressed by the following Equation (1).

$$\begin{matrix}[ Math . 1 ] & \\ {pc}_{i} ( {rec}_{i} , {imc}_{i} ) = {pc}_{i} ( \frac{{re}_{i} + res}{2} , \frac{{im}_{i} + ims}{2} ) & ( 1 )\end{matrix}$$

An I signal component recᵢ of the direct-current component pcᵢ expressed by (1) described above is subtracted from the I signal reᵢ of the first IQ information pᵢ, a Q signal component imcᵢ of the direct-current component pcᵢ is subtracted from the Q signal imᵢ of the first IQ information pᵢ, and thus a displacement component pᵢ′ in the subject of displacement acquisition can be obtained. The displacement component pᵢ′ in this subject of displacement acquisition is expressed as the coordinates pᵢ′(reᵢ′, imᵢ′) with the origin at coordinates pcᵢ′(recᵢ-recᵢ, imcᵢ-imcᵢ) (=p₀(0, 0)) in the complex plane. The coordinates pᵢ′(reᵢ′, imᵢ′) are the coordinates pᵢ′(reᵢ′, imᵢ′) of the third IQ information in the present disclosure. The coordinates pᵢ′(reᵢ′, imᵢ′) of the third IQ information are expressed by the following Equation (2). In the following description, the third IQ information defined by the coordinates pᵢ′(reᵢ′, imᵢ′) in the complex plane is also referred to as “third IQ information pᵢ′”.

$$\begin{matrix}[ Math . 2 ] & \\ {p}_{i}^{′} ( {re}_{i}^{′} , {im}_{i}^{′} ) = {p}_{i}^{′} ( {re}_{i} - {rec}_{i} , {im}_{i} - {imc}_{i} ) & ( 2 )\end{matrix}$$

The displacement calculation unit 13 calculates a displacement dᵢ of the subject of displacement acquisition by using the third IQ information pᵢ′ generated by the direct-current component removal unit 12. Specifically, the displacement calculation unit 13 obtains an argument of the third IQ information pᵢ′ generated by the direct-current component removal unit 12 to calculate a phase shift φᵢ. When the wavelength of the transmission frequency of the first device 1 (the frequency of the radiation wave Tx1) is λ, the displacement dᵢ of the subject of displacement acquisition is expressed by the following Equation (3).

$$\begin{matrix}[ Math . 3 ] & \\ {d}_{i} = \frac{{ϕ}_{i}}{2 π} λ & ( 3 )\end{matrix}$$

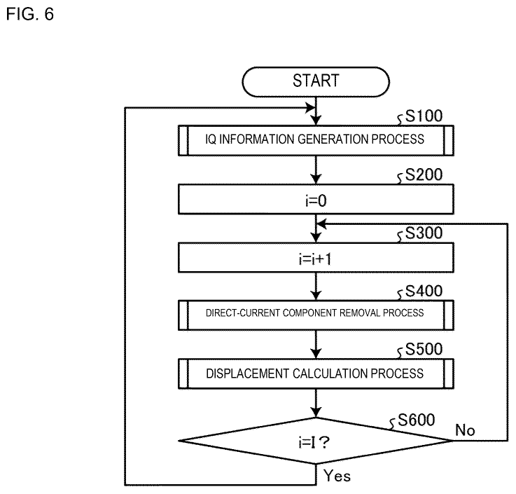

A specific example of a process in the radar system 100 according to the embodiment will be described below. FIG. 6 is a flowchart illustrating a specific example of a process in the radar system according to the embodiment. Here, an example will be described in which the phase shifter 22 of the second device 2 has the configuration of the first specific example illustrated in FIG. 2A. Furthermore, in the process to be described below, the first device 1 does not know the operation timing of the second device 2 in the second mode.

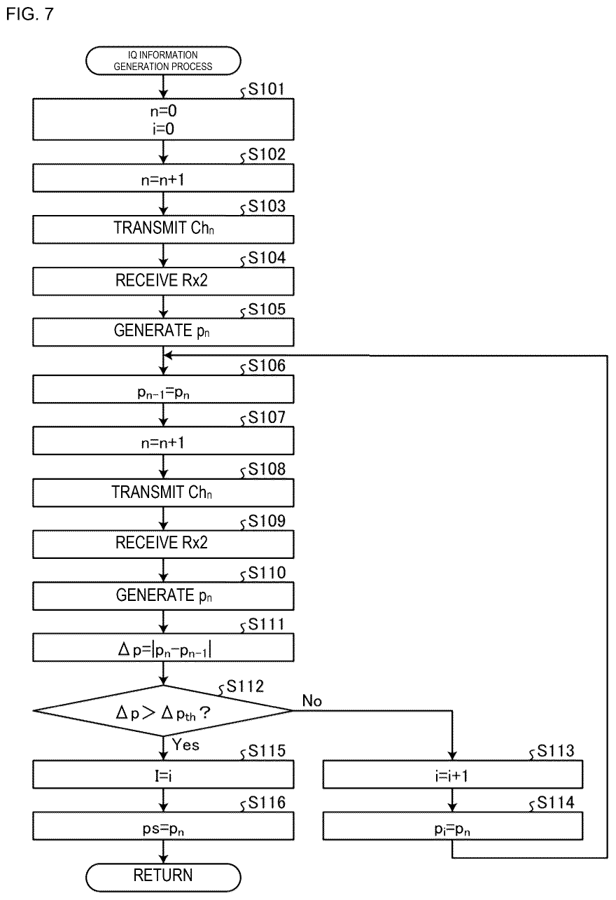

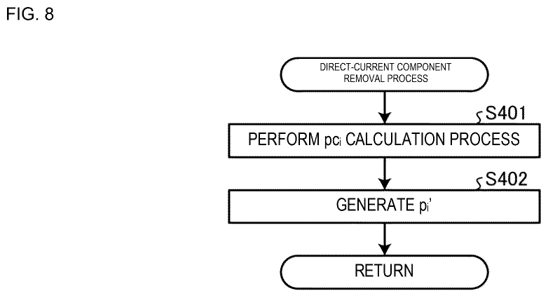

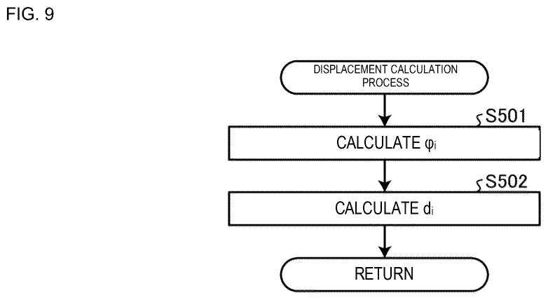

In the process illustrated in FIG. 6, the first device 1 performs an IQ information generation process (step S100) of generating first IQ information and second IQ information by using a reception wave Rx2, a direct-current component removal process (step S400) of generating third IQ information obtained by removing a direct-current component of the first IQ information by using the first IQ information and the second IQ information acquired by the IQ information generation process, and a displacement calculation process (step S500) of calculating a displacement of the subject of displacement acquisition by using the third IQ information generated by the direct-current component removal process. FIG. 7 is a sub-flowchart illustrating an example of the IQ information generation process. FIG. 8 is a sub-flowchart illustrating an example of the direct-current component removal process. FIG. 9 is a sub-flowchart illustrating an example of the displacement calculation process.

The first device 1 first performs the IQ information generation process illustrated in FIG. 7. The IQ information generation process is performed by the transmission/reception unit 11. Incidentally, IQ information is generated, for example, by existing filtering or FFT processing. The present disclosure is not to be limited by a method of generating IQ information.

As a precondition for the IQ information generation process illustrated in FIG. 7, the switch control unit 23 of the second device 2 sets the switch control signal Ssig at “H” in the reset period rst after receiving the chirp signal Chᵢ₊₁ (see FIG. 3A). Thus, the second device 2 operates in the first mode.

In the IQ information generation process illustrated in FIG. 7, the first device 1 first resets the number n of transmissions of the chirp signal Ch in the first mode, and the number i of receptions of the reception wave Rx2 before acquisition of second IQ information ps in the second mode (n=0, i=0, step S101), and increments the number n of transmissions of the chirp signal Ch in the first mode (n=n+1, step S102). The transmission/reception unit 11 transmits a chirp signal Chₙ (Ch₁ (see FIG. 3A and so forth)) (step S103).

The transmission/reception unit 11 receives a radio wave including a radiation wave Tx2 from the second device 2 (step S104). The transmission/reception unit 11 generates IQ information pₙ by using the reception wave Rx2 received in step S104 (step S105).

The first device 1 takes the IQ information pₙ as Pₙ₋₁ (pₙ₋₁=pₙ, step S106), and increments the number n of transmissions of the chirp signal Ch in the first mode (n=n+1, step S107). The transmission/reception unit 11 transmits a chirp signal Chₙ (step S108).

Subsequently, the transmission/reception unit 11 receives a reception wave Rx2 (step S109) and generates IQ information pₙ (step S110).

The transmission/reception unit 11 calculates a difference value Δp between the IQ information Pₙ and the IQ information pₙ₋₁ (Δp=|pₙ−pₙ₋₁|, step S111) and determines whether or not the difference value Δp is above a predetermined threshold pₜₕ (Δp>pₜₕ, step S112). When the difference value Δp is the predetermined threshold pₜₕ or less (Δp≤pₜₕ, No in step S112), the transmission/reception unit 11 increments the number i of receptions of the reception wave Rx2 before acquisition of second IQ information ps in the second mode (i=i+1, step S113), takes the IQ information pₙ as first IQ information pᵢ (pᵢ=pₙ, step S114), and outputs the first IQ information pᵢ to the subsequent-stage direct-current component removal unit 12. Then, the processing in step S106 and the subsequent steps are repeated.

When the difference value Δp exceeds the predetermined threshold pₜₕ (Δp>pₜₕ, Yes in step S112), the transmission/reception unit 11 takes the current number i of receptions of the reception wave Rx2 as the total number I of pieces of first IQ information pᵢ (I=i, step S115), takes the IQ information pₙ generated in last processing in step S110 as second IQ information ps (ps=pₙ, step S116), and outputs the second IQ information ps to the subsequent-stage direct-current component removal unit 12.

Here, the case where the difference value Δp is above the predetermined threshold pₜₕ (Δp>pₜₕ, Yes in step S112) indicates that the switch control unit 23 of the second device 2 has controlled the switch control signal Ssig from “H” to “L” in the reset period rstᵢ₊₁ after receiving the chirp signal Chᵢ. Thus, the second device 2 operates in the second mode. As a result, the IQ information pₙ generated by using the reception wave Rx2 changes significantly relative to the last value pₙ₋₁. In the present disclosure, when it is detected that the difference value Δp=|pₙ−pₙ₋₁|, which is the amount by which the IQ information pₙ has changed relative to the last value pₙ₋₁, has exceeded the threshold pₜₕ (Δp>pₜₕ, Yes in step S112), the IQ information pₙ generated in the last processing in step S110 is taken as the second IQ information ps (ps=pₙ, step S116).

Referring back to FIG. 6, the first device 1 resets the number i of the first IQ information pᵢ acquired by the transmission/reception unit 11 (i=0, step S200), increments the number i (i=i+1, step S300), and performs the direct-current component removal process (step S400). The direct-current component removal process is performed by the direct-current component removal unit 12.

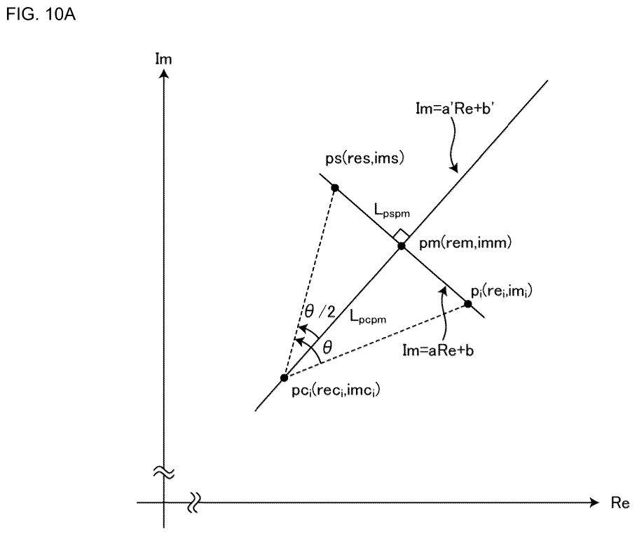

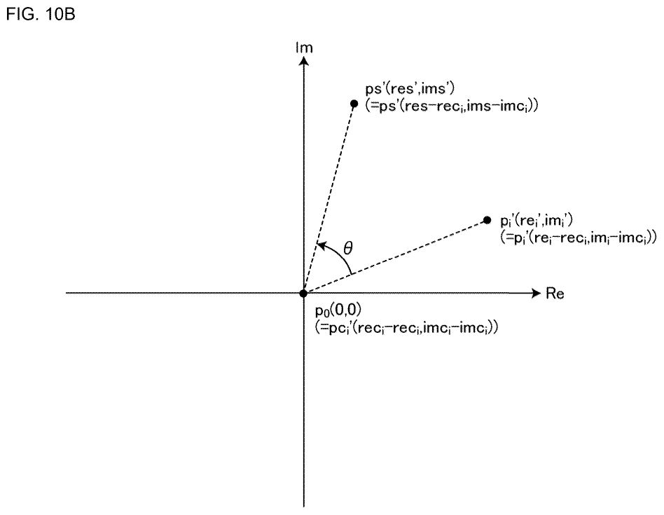

In the direct-current component removal process illustrated in FIG. 8, the direct-current component removal unit 12 first calculates a direct-current component pcᵢ of the first IQ information pᵢ (step S401). FIG. 10A is a first conceptual diagram illustrating a specific example of a direct-current component calculation process in a complex plane. In the complex plane, a line including the coordinates pᵢ(reᵢ, imᵢ) defining the first IQ information and the coordinates ps(res, ims) defining the second IQ information with slope a and intercept b is expressed by the following Equation (4).

$$\begin{matrix}[ Math . 4 ] & \\ Im = a Re + b & ( 4 )\end{matrix}$$

The slope a is expressed by the following Equation (5). In the following Equation (5), reᵢ denotes an I signal included in the first IQ information pᵢ, and imᵢ denotes a Q signal included in the first IQ information pᵢ. Furthermore, res denotes an I signal included in the second IQ information ps, and ims denotes a Q signal included in the second IQ information ps.

$$\begin{matrix}[ Math . 5 ] & \\ a = \frac{{im}_{i} - ims}{{re}_{i} - res} & ( 5 )\end{matrix}$$

In the complex plane, an I signal component rem of midpoint coordinates pm(rem, imm) between the coordinates pᵢ(reᵢ, imᵢ) defining the first IQ information and the coordinates ps(res, ims) defining the second IQ information is expressed by the following Equation (6), and a Q signal component imm is expressed by the following Equation (7).

$$\begin{matrix}[ Math . 6 ] & \\ rem = \frac{{re}_{i} + res}{2} & ( 6 )\end{matrix}$$

$$\begin{matrix}[ Math . 7 ] & \\ imm = \frac{{im}_{i} + ims}{2} & ( 7 )\end{matrix}$$

In the complex plane, the coordinates pcᵢ(recᵢ, imcᵢ) defining a direct-current component of the coordinates pᵢ(reᵢ, imᵢ) defining the first IQ information (and the coordinates ps(res, ims) defining the second IQ information) exist on a line passing through the midpoint coordinates pm(rem, imm) between the coordinates pᵢ(reᵢ, imᵢ) defining the first IQ information and the coordinates ps(res, ims) defining the second IQ information and orthogonal to the line including the coordinates pᵢ(reᵢ, imᵢ) defining the first IQ information and the coordinates ps(res, ims) defining the second IQ information. This line with slope a′ and intercept b′ is expressed by the following Equation (8).

$$\begin{matrix}[ Math . 8 ] & \\ Im = {a}^{′} Re + {b}^{′} & ( 8 )\end{matrix}$$

The slope a′ is expressed by the following Equation (9). Furthermore, the intercept b′ is expressed by the following Equation (10).

$$\begin{matrix}[ Math . 9 ] & \\ {a}^{′} = - \frac{{re}_{i} - res}{{im}_{i} - ims} & ( 9 )\end{matrix}$$

$$\begin{matrix}[ Math . 10 ] & \\ {b}^{′} = \frac{{im}_{i} + ims}{2} + \frac{{re}_{i}^{2} - {res}^{2}}{2 ( {im}_{i} - ims )} & ( 10 )\end{matrix}$$

Since the direct-current component coordinates pcᵢ(recᵢ, imcᵢ) are coordinates on the line expressed by Equation (8) described above, the following Equation (11) can be obtained. When the slope a′ expressed by Equation (9) described above and the intercept b′ expressed by Equation (10) described above are substituted in the following Equation (11), a relational equation expressed by the following Equation (12) can be obtained.

$$\begin{matrix}[ Math . 11 ] & \\ {imc}_{i} = {a}^{′} {rec}_{i} + {b}^{′} & ( 11 )\end{matrix}$$

$$\begin{matrix}[ Math . 12 ] & \\ {imc}_{i} = ( - \frac{{re}_{i} - res}{{im}_{i} - ims} ) rec + \frac{{im}_{i} + ims}{2} + \frac{{re}_{i}^{2} - {res}^{2}}{2 ( {im}_{i} - ims )} & ( 12 )\end{matrix}$$

Furthermore, when coordinates on the line expressed by Equation (8) described above are the direct-current component coordinates pcᵢ(recᵢ, imcᵢ), a line segment L_{pcpm} and a line segment Lₚₛₚₘ are expressed by the following Equation (13) by using the phase shift amount (amount of phase delay) θ in the phase shifter 22 of the second device 2. The line segment L_{pcpm} connects the direct-current component coordinates pcᵢ(recᵢ, imcᵢ) and the midpoint coordinates pm(rem, imm) between the coordinates pᵢ(reᵢ, imᵢ) defining the first IQ information and the coordinates ps(res, ims) defining the second IQ information. The line segment Lₚₛₚₘ connects the coordinates ps(res, ims) defining the second IQ information and the midpoint coordinates pm(rem, imm) between the coordinates pᵢ(reᵢ, imᵢ) defining the first IQ information and the coordinates ps(res, ims) defining the second IQ information.

$$\begin{matrix}[ Math . 13 ] & \\ \frac{{L}_{pspm}}{{L}_{pcpm}} = tan \frac{θ}{2} & ( 13 )\end{matrix}$$

The line segment L_{pcpm} is expressed by the following Equation (14), and the line segment Lₚₛₚₘ is expressed by the following Equation (15).

$$\begin{matrix}[ Math . 14 ] & \\ {L}_{pcpm}^{2} = {( rem - {rec}_{i} )}^{2} + {( imm - {imc}_{i} )}^{2} & ( 14 )\end{matrix}$$

$$\begin{matrix}[ Math . 15 ] & \\ {L}_{pspm}^{2} = {( res - rem )}^{2} + {( ims - imm )}^{2} & ( 15 )\end{matrix}$$

From Equations (13) to (15) described above, a relational equation expressed by the following Equation (16) can be obtained.

$$\begin{matrix}[ Math . 16 ] & \\ \frac{{( res + rem )}^{2} + {( ims - imm )}^{2}}{{( rem + {rec}_{i} )}^{2} + {( imm - {imc}_{i} )}^{2}} = {( tan \frac{θ}{2} )}^{2} & ( 16 )\end{matrix}$$

FIG. 10B is a first conceptual diagram illustrating coordinates of post-direct-current component removal first IQ information and second IQ information in a complex plane.

As illustrated in FIG. 10B, an I signal component reᵢ′ of post-direct-current component removal first IQ information pᵢ′ can be calculated by subtracting the I signal component recᵢ of the direct-current component pcᵢ from the I signal reᵢ of the first IQ information pᵢ. Furthermore, a Q signal component imᵢ′ of the post-direct-current component removal first IQ information pᵢ′ can be calculated by subtracting the Q signal component imcᵢ of the direct-current component pcᵢ from the Q signal of the first IQ information pᵢ.

Similarly, an I signal component res′ of post-direct-current component removal second IQ information ps′ can be calculated by subtracting the I signal component recᵢ of the direct-current component pcᵢ from the I signal res of the second IQ information ps. Furthermore, a Q signal component ims′ of the post-direct-current component removal second IQ information ps′ can be calculated by subtracting the Q signal component imcᵢ of the direct-current component pcᵢ from the Q signal ims of the second IQ information ps.

The coordinates ps′(res′, ims′) defining the second IQ information are coordinates delayed by the phase shift amount θ in the phase shifter 22 of the second device 2 with respect to the coordinates pᵢ′(reᵢ′, imᵢ′) defining the first IQ information, and thus the following Equations (17) and (18) can be obtained.

$$\begin{matrix}[ Math . 17 ] & \\ res - rem = ( {re}_{i} - {rec}_{i} ) cos θ - ( {im}_{i} - {imc}_{i} ) sin θ & ( 17 )\end{matrix}$$

$$\begin{matrix}[ Math . 18 ] & \\ ims - imm = ( {re}_{i} - {rec}_{i} ) sin θ + ( {im}_{i} - {imc}_{i} ) cos θ & ( 18 )\end{matrix}$$

The I signal component recᵢ and Q signal component imcᵢ of the direct-current component pcᵢ of the first IQ information pᵢ (and the second IQ information ps) can be calculated by using Equation (12) described above, and Equations (16) to (18) described above.

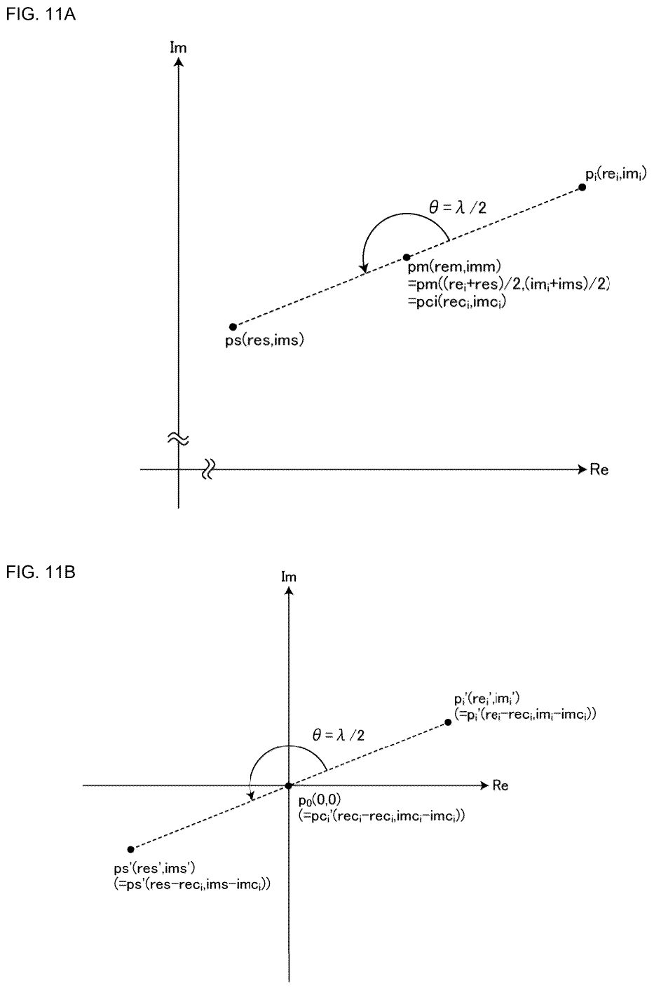

FIG. 11A is a second conceptual diagram illustrating a specific example of the direct-current component calculation process in a complex plane. FIG. 11B is a second conceptual diagram illustrating coordinates of post-direct-current component removal first IQ information and second IQ information in a complex plane.

FIGS. 11A and 11B illustrate a case where the phase shift amount (amount of phase delay) θ in the phase shifter 22 of the second device 2 is λ/2 (λ is the wavelength of the transmission frequency of the first device 1 (the frequency of the radiation wave Tx1)). In this case, as described above, the direct-current component pcᵢ can be calculated as a mean value of the first IQ information pᵢ and the second IQ information ps. At this time, the direct-current component coordinates pcᵢ(recᵢ, imcᵢ) in the complex plane are expressed by Equation (1) described above.

After the direct-current component pcᵢ calculation process (step S401), the direct-current component removal unit 12 subtracts the I signal component recᵢ of the direct-current component pcᵢ from the I signal reᵢ of the first IQ information pᵢ and subtracts the Q signal component imcᵢ of the direct-current component pcᵢ from the Q signal imᵢ of the first IQ information pᵢ to generate the third IQ information pᵢ′ expressed by Equation (2) described above (step S402), and outputs the third IQ information pᵢ′ to the subsequent-stage displacement calculation unit 13.

Referring back to FIG. 6, the first device 1 subsequently performs the displacement calculation process (step S500). The displacement calculation process is performed by the displacement calculation unit 13.

In the displacement calculation process illustrated in FIG. 9, the displacement calculation unit 13 first obtains an argument of the third IQ information pᵢ′ generated by the direct-current component removal unit 12 to calculate a phase shift φᵢ (step S501). Then, the displacement calculation unit 13 calculates a displacement dᵢ of the subject of displacement acquisition by using the wavelength λ of the transmission frequency of the first device 1 (the frequency of the radiation wave Tx1) in accordance with Equation (3) described above (step S502).

Referring back to FIG. 6, the first device 1 determines whether or not the number i of the first IQ information pᵢ acquired by the transmission/reception unit 11 has reached the total number I of pieces of first IQ information pᵢ (step S600). When the number i of the first IQ information pᵢ acquired by the transmission/reception unit 11 has not reached the total number I of pieces of first IQ information pᵢ (No in step S600), the first device 1 returns to the processing in step S300 and repeats the direct-current component removal process (step S400) and the displacement calculation process (step S500). When the number i of the first IQ information pᵢ acquired by the transmission/reception unit 11 reaches the total number I of pieces of first IQ information pᵢ, the first device 1 returns to step S100 and repeats the process illustrated in FIG. 6.

In a case where the subject of displacement acquisition is, for example, a human body, the above-described process enables the effect of a direct-current component included in a reflected-wave component from a stationary object or the like other than the human body to be reduced. This enables a minute displacement component, such as respiration or pulse, to be acquired with high accuracy.

### Modifications

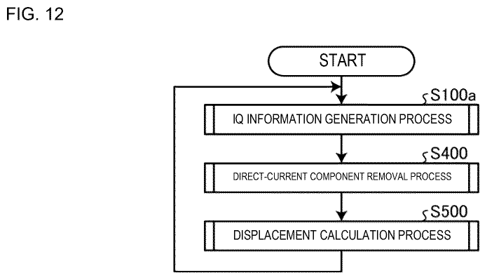

FIG. 12 is a flowchart illustrating a modification of the process in the radar system according to the embodiment. In the modification illustrated in FIG. 12, an example of processing for a case where the switching cycle of the switch control signal in the switch control unit 23 is twice the transmission cycle of the chirp signal Ch as illustrated in FIG. 3B is given. Note that the direct-current component removal process (step S400) and the displacement calculation process (step S500) are the same as those according to the above-described embodiment, and thus a description thereof is omitted. Here, an IQ information generation process (step S100a) according to the modification will be described.

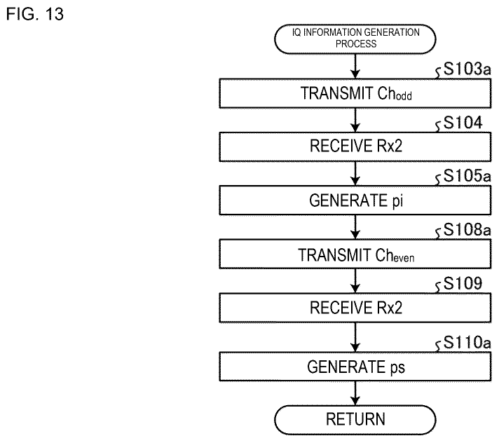

FIG. 13 is a sub-flowchart illustrating an example of the IQ information generation process according to the modification. As a precondition for the IQ information generation process according to the modification illustrated in FIG. 13, the switch control unit 23 of the second device 2 sets the switch control signal Ssig at “H” in a reset period rst_{odd} after receiving the chirp signal Chₑᵥₑₙ (see FIG. 3B). Thus, the second device 2 operates in the first mode.

In the IQ information generation process illustrated in FIG. 13, the transmission/reception unit 11 transmits a chirp signal Ch_{odd} (step S103a), receives a radio wave (reception wave Rx2) including a radiation wave Tx2 from the second device 2 (step S104), and generates first IQ information pᵢ by using the received radio wave (reception wave Rx2) (step S105a).

The switch control unit 23 of the second device 2 sets the switch control signal Ssig at “L” in a reset period rstₑᵥₑₙ after receiving the chirp signal Ch_{odd} (see FIG. 3B). Thus, the second device 2 operates in the second mode.

The transmission/reception unit 11 transmits a chirp signal Chₑᵥₑₙ (step S108a), receives a radio wave (reception wave Rx2) including a radiation wave Tx2 from the second device 2 (step S109), and generates second IQ information ps by using the received radio wave (reception wave Rx2) (step S110a).

Referring back to FIG. 12, the first device 1 performs the direct-current component removal process (step S400, see FIG. 8) by using the first IQ information pᵢ and the second IQ information ps acquired by the IQ information generation process according to the modification and performs the displacement calculation process (step S500, see FIG. 9) by using third IQ information pᵢ′ generated by the direct-current component removal process.

The switch control unit 23 of the second device 2 sets the switch control signal Ssig at “H” in the reset period rst_{odd} after receiving the chirp signal Chₑᵥₑₙ (see FIG. 3B). Thus, the second device 2 operates in the first mode. Then, the first device 1 repeats the process illustrated in FIG. 13.

As described above, the radar system 100 according to the embodiment and the modification includes the first device 1 that acquires a displacement of a subject of displacement acquisition by transmitting and receiving a radio wave, and the second device 2 that is installed at the subject of displacement acquisition in the radar system 100 and is disposed within a range where the second device 2 can transmit and receive a radio wave to and from the first device 1. The second device 2 has a first mode in which the second device 2 reradiates a received radio wave, and a second mode in which the second device 2 delays the phase of a received radio wave and reradiates the received radio wave. The first device 1 generates first IQ information (first information) pᵢ by using a radio wave received in a first period during which the second device 2 is operating in the first mode, and generates second IQ information (second information) ps by using a radio wave received in a second period during which the second device 2 is operating in the second mode. Subsequently, the first device 1 calculates a direct-current component pcᵢ of the first IQ information pᵢ by using the generated first IQ information pᵢ and the generated second IQ information ps, and generates third IQ information (third information) pᵢ′ obtained by removing the direct-current component pcᵢ from the first IQ information pᵢ. Thus, third IQ information (third information) pᵢ′ obtained by removing a direct-current component included in a reflected-wave component from a stationary object other than a target regarded as the subject of displacement acquisition in the radar system 100, or by removing a direct-current component corresponding to the static signal path between the first and second devices and reflections from stationary clutter, can be obtained.

Furthermore, the first device 1 calculates a displacement dᵢ of the subject of displacement acquisition by using the third IQ information (third information) pᵢ′ obtained by removing the direct-current component pcᵢ of the first IQ information pᵢ. This enables a minute displacement component of the subject of displacement acquisition to be acquired with high accuracy.

The present disclosure may take the following configuration as described above or in place of the above.
(1) A radar system according to an aspect of the present disclosure comprising: a first device configured to acquire a displacement of a subject of displacement acquisition by transmitting and receiving a radio wave; and a second device installed at the subject of displacement acquisition and disposed within a range where the second device can transmit and receive a radio wave to and from the first device, wherein the second device has a first mode in which the second device reradiates a received radio wave, and a second mode in which the second device delays a phase of a received radio wave and reradiates the received radio wave, and wherein the first device calculates, by using first information acquired in a first period during which the second device is operating in the first mode and second information acquired in a second period during which the second device is operating in the second mode, a direct-current component of the first information, and generates third information obtained by removing the direct-current component from the first information.

In this configuration, third information obtained by removing a direct-current component included in a reflected-wave component from a stationary object or the like other than the subject of displacement acquisition can be obtained.
(2) The radar system according to (1), wherein the first information and the second information are defined by coordinates in a complex plane.
(3) The radar system according to (2), wherein the first device calculates the direct-current component by using an amount of phase delay of a radio wave reradiated when the second device is operating in the second mode.

In this configuration, a direct-current component of the first information can be calculated by using the amount of phase delay of a radio wave reradiated when the second device is operating in the second mode, the first information acquired in the first period during which the second device is operating in the first mode, and the second information acquired in the second period during which the second device is operating in the second mode. Third information, from which a minute displacement component of the subject of displacement acquisition can be acquired with high accuracy, can be obtained by removing this direct-current component from the first information.
(4) The radar system according to (2), wherein, in the second mode, the second device delays a phase of a received radio wave by half a wavelength of a transmission frequency of the first device and reradiates the received radio wave.

In this configuration, a mean value of the first information and the second information can be calculated as the direct-current component of the first information.
(5) The radar system according to (4), wherein the first device calculates a mean value of the first information and the second information as the direct-current component.

In this configuration, the direct-current component of the first information is easy to calculate.
(6) The radar system according to (1) to (5), wherein the first device calculates a displacement of the subject of displacement acquisition by using the third information.

In this configuration, a highly accurate displacement component can be acquired in which the effect of a direct-current component included in a reflected-wave component from a stationary object or the like other than the subject of displacement acquisition is reduced.
(7) A signal processing method for a radar system according to an aspect of the present disclosure including a first device configured to acquire a displacement of a subject of displacement acquisition by transmitting and receiving a radio wave, and a second device installed at the subject of displacement acquisition and disposed within a range where the second device can transmit and receive a radio wave to and from the first device, the second device having a first mode in which the second device reradiates a received radio wave, and a second mode in which the second device delays a phase of a received radio wave and reradiates the received radio wave, the signal processing method comprising: with the first device, a first step of generating first information by using a radio wave received in a first period during which the second device is operating in the first mode; a second step of generating second information by using a radio wave received in a second period during which the second device is operating in the second mode; a third step of calculating a direct-current component of the first information by using the first information and the second information; and a fourth step of generating third information obtained by removing the direct-current component from the first information.

In this configuration, third information obtained by removing a direct-current component included in a reflected-wave component from a stationary object or the like other than the subject of displacement acquisition can be obtained.
(8) The signal processing method for the radar system according to (7), wherein the first information and the second information are defined by coordinates in a complex plane.
(9) The signal processing method for the radar system according to (8), wherein, in the third step, the first device calculates the direct-current component by using an amount of phase delay of a radio wave reradiated when the second device is operating in the second mode.

In this configuration, a direct-current component of the first information can be calculated by using the amount of phase delay of a radio wave reradiated when the second device is operating in the second mode, the first information acquired in the first period during which the second device is operating in the first mode, and the second information acquired in the second period during which the second device is operating in the second mode. Third information, from which a minute displacement component of the subject of displacement acquisition can be acquired with high accuracy, can be obtained by removing this direct-current component from the first information.
(10) The signal processing method for the radar system according to (8), wherein, in the second mode, the second device delays a phase of a received radio wave by half a wavelength of a transmission frequency of the first device and reradiates the received radio wave.

In this configuration, a mean value of the first information and the second information can be calculated as the direct-current component of the first information.
(11) The signal processing method for the radar system according to (10), wherein, in the third step, the first device calculates a mean value of the first information and the second information as the direct-current component.

In this configuration, the direct-current component of the first information is easy to calculate.
(12) The signal processing method for the radar system according to (7) to (11), further comprising, with the first device, a fifth step of calculating a displacement of the subject of displacement acquisition by using the third information.

In this configuration, a highly accurate displacement component can be acquired in which the effect of a direct-current component included in a reflected-wave component from a stationary object or the like other than the subject of displacement acquisition is reduced.

The present disclosure can provide the radar system that can acquire a minute displacement component of the subject of displacement acquisition with high accuracy, and the signal processing method for the same.

### REFERENCE SIGNS LIST

1 first device
2 second device
11 transmission/reception unit
12 direct-current component removal unit
13 displacement calculation unit
21 directional coupler
22 phase shifter
23 switch control unit

1. A radar system comprising:
a first device configured to acquire a displacement of a subject of displacement acquisition by transmitting and receiving a radio wave; and
a second device installed at the subject of displacement acquisition and disposed within a range where the second device can transmit and receive a radio wave to and from the first device,
wherein the second device has
a first mode in which the second device reradiates a received radio wave, and
a second mode in which the second device delays a phase of a received radio wave and reradiates the received radio wave, and
wherein the first device
calculates, by using first information acquired in a first period during which the second device is operating in the first mode and second information acquired in a second period during which the second device is operating in the second mode, a direct-current component of the first information, and generates third information obtained by removing the direct-current component from the first information.

2. The radar system according to claim 1,
wherein the first information and the second information are defined by coordinates in a complex plane.

3. The radar system according to claim 2,
wherein the first device
calculates the direct-current component by using an amount of phase delay of a radio wave reradiated when the second device is operating in the second mode.

4. The radar system according to claim 2,
wherein, in the second mode, the second device
delays a phase of a received radio wave by half a wavelength of a transmission frequency of the first device and reradiates the received radio wave.

5. The radar system according to claim 4,
wherein the first device
calculates a mean value of the first information and the second information as the direct-current component.

6. The radar system according to claim 1,
wherein the first device calculates a displacement of the subject of displacement acquisition by using the third information.

7. The radar system according to claim 1,
wherein the first device is a Frequency Modulated Continuous Wave (FMCW) radar that transmits a plurality of chirp signals.

8. The radar system according to claim 7,
wherein the first period corresponds to a first set of one or more chirp signals and the second period corresponds to a second, subsequent chirp signal, and wherein the first device is configured to identify the second period by detecting a difference value between IQ information from consecutive chirp signals that exceeds a predetermined threshold.

9. The radar system according to claim 7,
wherein the second device is configured to alternate between the first mode and the second mode for successive chirp signals, such that odd-numbered chirp signals are received during the first period and even-numbered chirp signals are received during the second period.

10. A signal processing method for a radar system including a first device configured to acquire a displacement of a subject of displacement acquisition by transmitting and receiving a radio wave, and a second device installed at the subject of displacement acquisition and disposed within a range where the second device can transmit and receive a radio wave to and from the first device,
the second device having
a first mode in which the second device reradiates a received radio wave, and
a second mode in which the second device delays a phase of a received radio wave and reradiates the received radio wave,
the signal processing method comprising: with the first device,
generating first information by using a radio wave received in a first period during which the second device is operating in the first mode;
generating second information by using a radio wave received in a second period during which the second device is operating in the second mode;
calculating a direct-current component of the first information by using the first information and the second information; and
generating third information obtained by removing the direct-current component from the first information.

11. The signal processing method for the radar system according to claim 10,
wherein the first information and the second information are defined by coordinates in a complex plane.

12. The signal processing method for the radar system according to claim 11,
wherein, in calculating the direct-current component, the first device
calculates the direct-current component by using an amount of phase delay of a radio wave reradiated when the second device is operating in the second mode.

13. The signal processing method for the radar system according to claim 11,
wherein, in the second mode, the second device
delays a phase of a received radio wave by half a wavelength of a transmission frequency of the first device and reradiates the received radio wave.

14. The signal processing method for the radar system according to claim 10,
wherein, in calculating the direct-current component, the first device
calculates a mean value of the first information and the second information as the direct-current component.

15. The signal processing method for the radar system according to claim 10,
wherein the first device is a Frequency Modulated Continuous Wave (FMCW) radar that transmits a plurality of chirp signals.

16. The signal processing method for the radar system according to claim 10, further comprising,
with the first device,
calculating a displacement of the subject of displacement acquisition by using the third information.

17. The signal processing method for the radar system according to claim 10, wherein calculating the displacement of the subject include:
calculating a phase shift ϕi from an argument of the third information; and
calculating the displacement, di, based on the phase shift ϕi and a wavelength, λ, of the transmitted radio wave, according to the equation:
$${d}_{i} = \frac{{ϕ}_{i}}{2 π} λ .$$

18. A device for use with a radar system, the device configured to be installed at a subject of displacement acquisition, the device comprising:
an antenna configured to receive a first radio wave from the radar system and to reradiate a second radio wave toward the radar system;
a phase shifter coupled to the antenna; and
a switch control circuit configured to operate the phase shifter in:
a first mode, wherein the second radio wave is reradiated; and
a second mode, wherein the phase shifter introduces a predetermined phase delay, causing the second radio wave to be reradiated with the predetermined phase delay having a different phase delay than the reradiated second radio wave.

19. The device according to claim 18, wherein the phase shifter includes:
a first signal path;
a second signal path including a phase delay element; and
at least one switch controlled by the switch control circuit to direct the first radio wave through either the first signal path for operation in the first mode or the second signal path for operation in the second mode.

20. The device according to claim 19, wherein the predetermined phase delay is half a wavelength of a transmission frequency of the radar system.
